(19) United States
(12) Patent Application Publication
(10) Pub. No.: US 2022/0336062 A1
(43) Pub. Date: Oct. 20, 2022

(54) APPARATUS AND METHOD FOR PROVIDING MEDICAL INSTITUTION INFORMATION USING NON-REIMBURSEMENT INFORMATION

(71) Applicant: Hoijun PARK, Gwangyang-si (KR)

(72) Inventor: Hoijun PARK, Gwangyang-si (KR)

(21) Appl. No.: 17/638,656

(22) PCT Filed: May 25, 2020
(86) PCT No.: PCT/KR2020/006759

§ 371 Date: Feb. 25, 2022

(30) Foreign Application Priority Data
Aug. 27, 2019 (KR) 10-2019-0105289

(51) Int. Cl. G16H 10/60; G16H 20/00

(52) U.S. Cl. CPC G16H 10/60; G16H 20/00

(57) ABSTRACT

Disclosed is an apparatus and method for providing medical institution information using non-reimbursement information, which is the technical concept of the present invention. According to one embodiment of the present disclosure, the apparatus may comprise: a medical expense database established by including medical institution information, and reimbursement information and non-reimbursement information for medical expenses for items of medical services performed by a medical institution, which are obtained by scanning a medical expense receipt that a first user received; an input unit for receiving, from a second user, items of medical services to be performed; and an output unit which searches for the inputted items of medical services in the medical expense database, and if the items correspond to the non-reimbursement information, outputs a list of medical institutions that provide medical services for the inputted items of medical services corresponding to the non-reimbursement information.

### TECHNICAL FIELD

The present disclosure relates to an apparatus and method for providing medical institution information using non-reimbursement information. Specifically, the present disclosure may provide an apparatus and method for providing medical institution information using non-reimbursement information that is capable of providing fair medical services.

### BACKGROUND ART

When a user uses a medical service at a medical institution, the user is issued a paper medical expense receipt. The paper medical expense receipt records medical expenses divided into reimbursement information and non-reimbursement information for medical services. The user may check items of medical expenses recorded on the paper medical expense receipt to identify whether the medical service used by the user corresponds to reimbursement information or non-reimbursement information. Such a conventional medical service is not only provided to an individual user, but the medical expense information may also remain as personalized information. Personalized information may be good for information security but may act as an obstacle to providing generalized and fair medical services.

### DISCLOSURE

### Technical Problem

Therefore, it is an object of the disclosure to provide an apparatus and method for providing medical institution information using non-reimbursement information that allow use of a medical service of a medical institution by sharing information available as shared information among pieces of information included in a medical expense receipt. Specifically, the present disclosure may provide an apparatus and method for providing medical institution information using non-reimbursement information that allow a user to use a fair medical service.

The technical objectives of the present disclosure are not limited to the above, and other objectives described above may become apparent to those of ordinary skill in the art based on the following description.

According to the first aspect of the present disclosure, there is provided an apparatus for providing medical institution information using non-reimbursement information, the apparatus including: a medical expense database constructed to include reimbursement information and non-reimbursement information for medical expenses for items of medical service, which is performed by a medical institution, and medical institution information which are acquired by scanning a medical expense receipt received by a first user; an input unit configured to receive an item of medical service to be performed that is input from a second user; and an output unit configured to search for the input item of medical service in the medical expense database, and when the found item of medical service corresponds to the non-reimbursement information, output a list of medical institutions that provide a medical service for the input item of medical service corresponding to the non-reimbursement information.

The output unit may include one or more of a hospital and a pharmacy in the list of medical institutions.

The medical expense database may include a scan unit configured to acquire the reimbursement information and the non-reimbursement information from a paper medical expense receipt or an electronic medical expense receipt acquired by the first user and configured to store the acquired reimbursement information and non-reimbursement information.

The scan unit may include one or more of a camera or a communication unit.

The input unit and the output unit may be formed as a single embedded terminal.

The medical expense database may calculate a discrimination according to a difference in medical expenses between users for a same medical service item at a same medical institute and may store the discrimination, and the output unit outputs the list of medical institutions to further include the discrimination.

The output unit may output the list of medical institutions that are arranged in consideration of one or more of user location information, medical institution location information, and medical expense information.

According to the second aspect of the present disclosure, there is provided a method of providing medical institution information using non-reimbursement information, the method including: constructing a medical expense database to include reimbursement information and non-reimbursement information for medical expenses for items of medical service, which is performed by a medical institution, and medical institution information which are acquired by scanning a medical expense receipt received by a first user; receiving, by an input unit, an item of medical service to be performed, which is input from a second user; and searching for, by an output unit, the input item of medical service in the medical expense database and, when the found item of medical service corresponds to the non-reimbursement information, outputting a list of medical institutions that provide a medical service for the input item of medical service corresponding to the non-reimbursement information.

### Advantageous Effects

According to an embodiment of the present disclosure, a user shares information included in a medical expense receipt so that users including the user can use a fair medical service.

Users use fair medical services together so that the reliability of medical services can be increased.

It should be understood that the advantageous effects of the present disclosure are not limited to the above, and all other effects that may be deduced from constructions disclosed in the specification or the scope of claims of the present disclosure are included therein.

### DESCRIPTION OF DRAWINGS

FIG. 1 is an exemplary diagram illustrating an apparatus for providing medical institution information according to an embodiment.

FIG. 2 is an exemplary diagram illustrating an operation scenario of a medical institution information service according to an embodiment.

FIG. 3 is an operation flowchart showing a method of providing medical institution information according to an embodiment.

FIG. 4 is an exemplary diagram illustrating a system for providing medical institution information according to an embodiment.

### MODE OF THE DISCLOSURE

Although terms used herein are selected from among general terms that are currently and widely used in consideration of functions in the exemplary embodiments, these may be changed according to intentions or customs of those skilled in the art or the advent of new technology. However, when a specified term is defined and used in an arbitrary sense, a meaning of the term will be described in the specification in detail. Accordingly, the terms used herein are not to be defined as simple names of the components but should be defined based on the actual meaning of the terms and the whole context throughout the present specification.

Throughout the specification, the term “comprises” or “includes” and/or “comprising” or “including” means that one or more other components may not be further excluded unless context dictates otherwise. In the specification, the term “part” or “module” refers to a unit for processing at least one function or operation that may be implemented in hardware, software, or a combination thereof.

In the following, embodiments of the present disclosure will be described in detail with reference to the accompanying drawings. However, the present disclosure may be embodied in various ways and is not to be construed as limited to the embodiments set forth herein.

Hereinafter, embodiments of the present disclosure will be described in detail with reference to the accompanying drawings.

An apparatus for providing medical institution information provides a user, when a medical service requested by the user corresponds to non-reimbursement information, with a list of medical institutions that provide the medical service corresponding to non-reimbursement information, to allow the user to select a desired medical institution by referring to the list. For example, when a user desires to have a magnetic resonance imaging (MRI) diagnosis, and inputs a request for an MRI diagnosis service to the apparatus for providing medical institution information, the apparatus for providing medical institution information may display, on a display, a list of medical institutions that provide an MRI diagnosis service, which corresponds to non-reimbursement information, in consideration of one or more of a user location, a medical institution location, and medical expenses. The user selects a medical institution that meets a desired condition by referring to the list of medical institutions displayed on the display.

FIG. 1 is an exemplary diagram illustrating an apparatus for providing medical institution information according to an embodiment.

Referring to FIG. 1, an apparatus 100 for providing medical institution information includes a scan unit 110 that separately scans reimbursement information and non-reimbursement information for medical services from a paper medical expense receipt or electronic medical expense receipt; a medical expense database 200 that stores medical expense information corresponding to the scanned reimbursement information and non-reimbursement information therein; an input unit 120 that receives a medical service desired and input by a user; and an output unit 130 that searches for the input medical service in the medical expense database 200 and outputs a list of medical institutions for the medical service which corresponds to non-reimbursement information.

However, it should be understood by those skilled in the art that general-purpose components other than those shown in FIG. 1 may be further included in the apparatus 100 for providing medical institution information. For example, the output unit 130 may further include a display. Alternatively, it should be understood by those skilled in the art that some of the components shown in FIG. 1 may be omitted according to another embodiment.

The apparatus 100 for providing medical institution information may be implemented in a smartphone. The apparatus 100 for providing medical institution information 100 may be formed as an application and installed in a storage space of the smartphone such that the smartphone operates as the apparatus 100 for providing medical institution information. When a user executes an application for providing medical institution information, the smartphone operates as the apparatus 100 for providing medical institution information, a camera mounted on the smartphone may operate as the scan unit 110, and a display of the smartphone may operate as the output unit 130.

As a flaw of the medical service, the same medical institution may charge different medical expenses between users for the same medical service. In order to notify the user of such a flaw of the medical service, the output unit 130 may calculate the discrimination when it is found that the medical expense database 200 charges different medical expenses between users for the same medical service at the same medical institution. The discrimination may be calculated by considering one or more variables among the number of medical items of a medical institution, the difference in medical expenses between users, and initial medical expenses, and applying weights to the variables. For example, the value of the discrimination may be calculated by a relational expression of first weight×the number of medical items+second weight×difference in medical expenses+third weight×initial medical expenses. In this case, the first weight is a proportional constant of the number of medical items that may increase in proportion to the difference in medical expenses, the second weight is a proportional constant of the difference in medical expenses that may increase in proportion to the initial medical expenses, and the three-weighted value is a proportional constant of the initial medical expenses that may increase in proportion to the number of medical items and the difference in medical expenses.

The output unit 130 may output the list of medical institutions to further include the value of the discrimination, and the user may determine the reliability of the medical institution by referring to the discrimination value. The output unit 130 may output a reliability value instead of outputting the discrimination value so that the user may easily recognize the reliability. Here, the discrimination value is inversely proportional to the reliability value.

The apparatus 100 for providing medical institution information may be implemented as an embedded device, and the embedded device may include a smartphone, and as basic components, include a processor, a memory, and a display, and have a commercial operating system (OS) so that the apparatus 100 for providing medical institution information is implemented in the form of an application that operates on the OS. For example, in the case of a smartphone-medical institution information providing apparatus, the processor of the smartphone may operate as the scan unit 110, the input unit 120, and the output unit 130, and the display of the smartphone may operate as the output unit 130. From the perspective of an application, the software structure may include the scan unit 110, the input unit 120, and the output unit 130. The application may be constructed by an integrated development environment using a programming language according to various operating environments required by embedded devices.

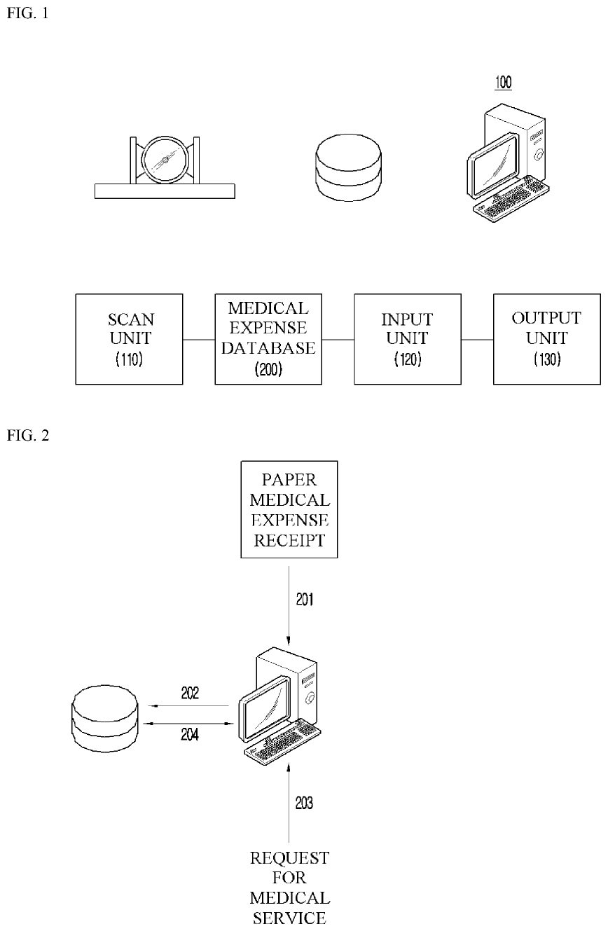

FIG. 2 is an exemplary diagram illustrating an operation scenario of a medical institution information service according to an embodiment.

The operation scenario of the medical institution information service may be described according to a flow of data transmitted and received between a medical expense database and an apparatus for providing medical institution information. The apparatus for providing medical institution information may transmit information related to reimbursement information and non-reimbursement information for medical services acquired from a paper medical expense receipt or electronic medical expense receipt to the medical expense database (201) to store the information in the medical expense database (202). The medical expense database may collect the information related to reimbursement information and non-reimbursement information for medical services from a plurality of apparatuses for providing medical institution information, and store the collected pieces of information in a storage space. When the apparatus for providing medical institution information receives a request for a medical service input from a user (203), the apparatus for providing medical institution information may access the medical expense database to receive a list of medical institutions corresponding to the input medical service (204). The apparatus for providing medical institution information may search for the list of medical institutions in the medical expense database using a search function.

In the operation scenario, a first user may be provided with medical services from a medical institution, receive a paper medical expense receipt, have the paper medical expense receipt scanned by an apparatus for providing medical institution information, and accumulate points. The accumulated points may be used to pay medical expenses when the first user uses medical services at a medical institution. When a second user operates an application for providing medical institution information mounted in a smartphone and inputs a medical service desired to use, an apparatus for providing medical institution information owned by the second user may output a list of medical institutions together with the reliability values. The second user may select a medical institution having a high reliability value and ranked higher.

In the operation scenario, the first user may use the points accumulated by scanning the paper medical expense receipt at a pharmacy among medical institutions. In this case, the pharmacy, which is a medical institution, has subscribed to a medical institution information providing service platform. A service environment may be established such that in a situation in which hospitals or pharmacies, which are various types of medical institutions, have subscribed to a medical institution information providing service platform, to which a first user and a second user also have subscribed, other users who subscribe to the platform may use the medical institution information providing service and use points accumulated when using medical services at a medial institution that is a member company.

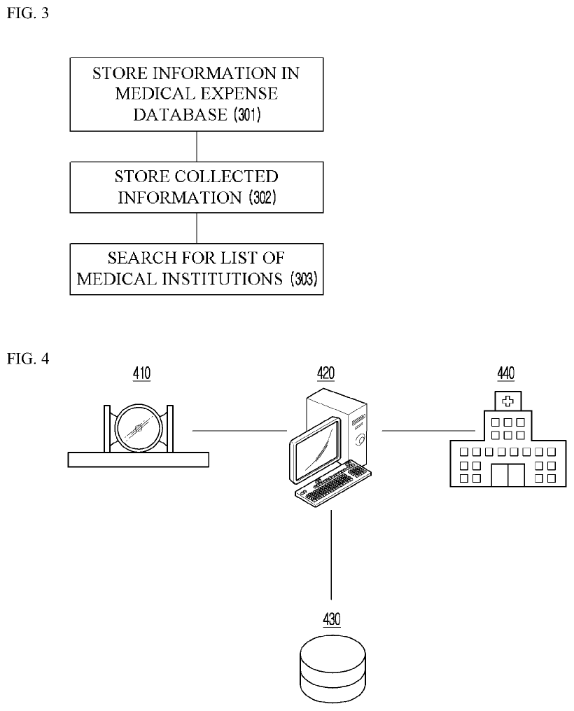

FIG. 3 is an operation flowchart showing a method of providing medical institution information according to an embodiment.

In operation 301, the apparatus for providing medical institution information may transmit information related to reimbursement information and non-reimbursement information of medical services acquired from a paper medical expense receipt or electronic medical expense receipt to the medical expense database to store the information in the medical expense database.

In operation 302, the medical expense database may collect the information related to reimbursement information and non-reimbursement information of medical services from a plurality of apparatuses for providing medical institution information and store the collected pieces of information in a storage space.

In operation 303, when the apparatus for providing medical institution information receives a request for a medical service input from a user, the apparatus for providing medical institution information may access the medical expense database to receive a list of medical institutions corresponding to the input medical service. The apparatus for providing medical institution information may search for the list of medical institutions in the medical expense database using a search function.

FIG. 4 is an exemplary diagram illustrating a system for providing medical institution information according to an embodiment.

Referring to FIG. 4, the system for providing medical institution information may include a scanning device 410 that scans reimbursement information and non-reimbursement information for medical services from a paper medical expense receipt or electronic medical expense receipt; a medical expense database 430 that stores information related to the reimbursement information and non-payment information for medical services scanned by the scanning device 410; an apparatus 420 for providing medical institution information that searches for a medical service input by a user in the medical expense database 430 and provides a list of medical institutions that provide the medical service corresponding to the non-reimbursement information; and a medical institution 440 that provides medical services.

However, it should be understood by those skilled in the art that general-purpose components other than those shown in FIG. 4 may be further included in the apparatus 420 for providing medical institution information. For example, the apparatus 420 for providing medical institution information may further include a display, a memory, and the like. Alternatively, it should be understood by those skilled in the art that some of the components shown in FIG. 4 may be omitted according to another embodiment.

Meanwhile, the method described above may be written in a program executable by computers and may be implemented on a general digital computer that operates the program using a computer readable media. A data structure used in the method may be recorded on a computer readable recoding medium through various methods. The computer readable recording media may include a magnetic recording medium (e.g., a read only memory (ROM), a random-access memory (RAM), a Universal Serial Bus (USB), a floppy disk, a hard disk, etc.), an optical reading medium (e.g., a compact disk (CD)-ROM, a digital video disk (DVD), etc.), and other recording media.

Those of ordinary skill in the technical field to which the present disclosure pertains should be able to understand that various modifications and alterations can be made without departing from the technical spirit or essential features of the present disclosure. Therefore, it should be understood that the disclosed methods are not limiting but illustrative in all aspects. The scope of the present disclosure is defined not by the above description but by the following claims, and it should be understood that all changes or modifications derived from the scope and equivalents of the claims fall within the scope of the present disclosure.

1. An apparatus for providing medical institution information using non-reimbursement information, the apparatus comprising:
a medical expense database constructed to include reimbursement information and non-reimbursement information for medical expenses for items of medical service, which is performed by a medical institution, and medical institution information which are acquired by scanning a medical expense receipt received by a first user;
an input unit configured to receive an item of medical service to be performed that is input from a second user; and
an output unit configured to search for the input item of medical service in the medical expense database, and when the found item of medical service corresponds to the non-reimbursement information, output a list of medical institutions that provide a medical service for the input item of medical service corresponding to the non-reimbursement information.

2. The apparatus of claim 1, wherein the output unit includes one or more of a hospital and a pharmacy in the list of medical institutions.

3. The apparatus of claim 1, wherein the medical expense database includes a scan unit configured to acquire the reimbursement information and the non-reimbursement information from a paper medical expense receipt or an electronic medical expense receipt acquired by the first user and configured to store the acquired reimbursement information and non-reimbursement information.

4. The apparatus of claim 3, wherein the scan unit includes one or more of a camera or a communication unit.

5. The apparatus of claim 1, wherein the input unit and the output unit are formed as a single embedded terminal.

6. The apparatus of claim 1, wherein the medical expense database calculates a discrimination according to a difference in medical expenses between users for a same medical service item at a same medical institute and stores the discrimination, and the output unit outputs the list of medical institutions to further include the discrimination.

7. The apparatus of claim 1, wherein the output unit outputs the list of medical institutions that are arranged in consideration of one or more of user location information, medical institution location information, and medical expense information.

8. A method of providing medical institution information using non-reimbursement information, the method comprising:
constructing a medical expense database to include reimbursement information and non-reimbursement information for medical expenses for items of medical service, which is performed by a medical institution, and medical institution information which are acquired by scanning a medical expense receipt received by a first user;
receiving, by an input unit, an item of medical service to be performed, which is input from a second user; and
searching for, by an output unit, the input item of medical service in the medical expense database and, when the found item of medical service corresponds to the non-reimbursement information, outputting a list of medical institutions that provide a medical service for the input item of medical service corresponding to the non-reimbursement information.
